(19) United States
(12) Patent Application Publication
(10) Pub. No.: US 2002/0197689 A1
(43) Pub. Date: Dec. 26, 2002

(54) Insecticidal peptides and methods for use of same

(76) Inventors: Gerardo Corzo, Osaka (JP); Pierre Escoubas, Valbonne (FR)

Correspondence Address: SUTHERLAND ASBILL & BRENNAN LLP, 999 PEACHTREE STREET, N.E., ATLANTA, GA, 30309, US

(21) Appl. No.: 09/814,452

(22) Filed: Mar. 22, 2001

Related U.S. Application Data
(60) Provisional application No. 60/191,380, filed on Mar. 22, 2000.

(51) Int. Cl. C12N 9/00; A61K 38/00

(52) U.S. Cl. 435/183; 424/94.1

(57) ABSTRACT

This invention relates to the purification of a group of insecticidally effective toxin peptides isolated from the venoms of the Paracoelotes and Xysticus species of spiders. The toxin peptides are characterized by their neurotoxic effect on insect pest and potential for little, if any, toxicity in mammals. Coding polynucleotide sequences for the toxin peptides are described, as are methods and vectors for production and targeted delivery of recombinant toxin peptides. Methods for the use of the toxin peptides as insecticides are also provided.

### BACKGROUND OF THE INVENTION

1. Field of the Invention

The invention relates to agents for use in the biological control of insect populations. In particular, the invention relates to spider venom peptides with insecticidal properties.

2. History Of The Related Art

A significant percentage of worldwide agricultural production is lost to insect damage each year. In addition, many serious human and animal diseases, including malaria, yellow fever, sleeping sickness, viral encephalitis, and plague, are transmitted by insects.

Synthetic chemical insecticides (such as chlorinated hydrocarbons (e.g., DDT), carbamates (e.g., carbaryl), organophosphates (e.g., malathion), synthetic pyrethroids (e.g., cypermethrin), insect growth regulators (e.g., diflubenzuron and methoprene) and metabolic disrupters (e.g., hydroxymethylnon)) are effective for controlling pest insects. However, the long-term environmental effects of such chemical pesticides, coupled with the development of resistance in many insect populations, have made the discovery of alternative agents for the biological control of insects an important goal for the pest control industry.

Insect pathogens and toxins are natural candidates for development for use in pest control. However, these products tend to be less potent and more difficult to manufacture than many chemical insecticides. As such, they represent only a small fraction of the presently available insecticide arsenal.

### SUMMARY OF THE INVENTION

The invention provides polypeptides derived from the venom of two species of spiders that possess potent insecticidal activity (“SV toxin peptides”) and are readily susceptible to manufacture, yet pose relatively little risk of toxicity toward non-insect species. More particularly, the present invention includes an isolated spider venom (SV) toxin peptide comprising a cysteine rich toxin motif, wherein the peptide is a Paracoelotes sp. peptide or a Xysticus acerbus peptide. For those peptides derived from Paracoelotes sp., the cysteine rich toxin motif is preferably a Pasp cysteine rich toxin motif as described below and more preferably comprises an amino acid sequence as shown in SEQ ID NO:21.

Also within the scope of the invention are polynucleotides (“SV toxin polynucleotides”) that encode SV toxin peptides and recombinant expression vectors, such as insect-pathogen viruses, for expression and targeted delivery of recombinant SV toxin peptides.

SV toxin peptides have been identified as having potential ion channel specificity within the insect nervous system, based on their lack of toxic effect on the nervous system of mice and their sequence homology to other peptides having such specificity. Functionally, SV toxin peptides are preferably selective for insect species, thereby minimizing the risk of adverse consequences from use of the peptides in applications that could bring them into contact with non-insect species.

Thus, in one respect, the invention consists of SV toxin peptides, as well as active fragments, analogs and derivatives thereof. For purposes of this disclosure, the term “SV toxin peptides” should be regarded as including active fragments, analogs and derivatives thereof, unless context otherwise requires. Preferred SV toxin peptides comprise amino acid sequences as shown in SEQ ID NO:1, SEQ ID NO:2, SEQ ID NO:3, SEQ ID NO:4 and SEQ ID NO:5. The SV toxin peptide of the present invention can further comprise signal peptides. Insecticide compositions comprising a carrier and SV toxin peptides are also provided.

In another respect, the invention consists of nucleic acid molecules that encode SV toxin peptides, as well as poly- and oligo-nucleotides that are specifically hybridizable to such nucleic acid molecules. Preferred SV toxin nucleic acids comprise amino acid sequences as shown in SEQ ID NO:6, SEQ ID NO:7, SEQ ID NO:8, SEQ ID NO:9 and SEQ ID NO:10.

The invention further consists of recombinant expression vectors including, without limitation, insect-pathogen viruses (e.g., baculovirus) expression vectors for the expression and targeted delivery of nucleic acid molecules encoding SV toxin peptides.

The invention also consists of methods for utilizing SV toxin peptides and SV nucleic acid molecules to identify other insecticides. For example, the SV toxin peptides of the invention are useful in the identification of their molecular target and of competitors for binding thereto. The nucleic acid molecules of the invention are useful, for example, to probe libraries to identify nucleotide sequences that might encode similar toxins produced from other organisms by using certain regions, e.g., conserved sequence regions, of the nucleotide sequence.

For use in the biological control of insect populations, the invention further provides methods for delivery of SV toxin peptides to insects directly and indirectly by, for example, targeted application of SV toxin peptides to insect populations, especially in sensitive environments, such as growing crops.

### DESCRIPTION OF THE DRAWINGS

FIG. 1 is graph of the results of the experiment in Example 1 for fractionation of Paracoelotes sp. Crude venom by reverse-phase HPLC.

FIG. 2 depicts the results from Example 1 when applying cation-exchange chromatography to the peak labeled “PaspI” in FIG. 1.

FIG. 3 depicts the further separation by reverse-phase chromatography of the peak labeled “PaspI” in FIG. 2.

FIGS. 4a-b show the amino acid and nucleotide sequences of PaspI, PaspII, PaspIII, PaspIV and XacI.

FIGS. 5a-c show the amino acid and nucleotide sequence of PaspI, PaspII, PaspIII, PaspIV and XacI operably linked to a signal sequence.

FIG. 6 is a table showing a comparison of the SV Paracoelotes sp. toxins of the present invention with toxins from A. aperta and H. curta.

FIG. 7 is a table showing the percentage of identical residues between the SV Paracoelotes sp. toxins of the present invention and toxins from A. aperta and H. curta as calculated for maximum homology.

FIG. 8 is a table showing a comparison of the insecticidal activity of Paracoelotes sp., A. aperta and H. curta crude venoms against S. litura.

FIG. 9 is a table showing a comparison of the insecticidal activities of the SV Paracoelotes sp. toxins of the present invention and Androctonus australis toxin against S. litura.

FIG. 10 is a table showing the effect of the PaspI, PaspII, PaspIII and PaspIV toxins on mice.

FIG. 11 is a table showing an identity comparison between PaspI, XacI and 1₅A.

### DETAILED DESCRIPTION OF THE INVENTION

Structural and Functional Characteristics of SV Toxin Peptides

The SV toxin peptides of the invention each have “insecticidal activity”. As used herein, the term “insecticidal activity” refers to an ability to interfere with the normal biochemical and physiological processes of an insect and to thereby cause the disablement or death of the insect. In one embodiment, the SV toxin peptides interfere with or inhibit the insect's neural functions. In a further embodiment, the SV toxin peptides bind to and interfere with ion channels in the insect. The term “insecticidal effect” is also used herein to refer to the disablement of death of the insect. Unless otherwise noted, and within the detectable parameters of the bioassays for such activity described herein, the insecticidal activity of SV toxin peptides does not extend to non-insect species or, if there is some effect on such species, the effect falls short of permanent impairment or mortality. It is also to be understood that the term “protein” and “peptide” are used interchangeably herein.

Each toxin includes an insecticidally active peptide derived from the venom of a spider; including, but not limited to, the venom of either Paracoelotes sp. (“Pasp” peptides) or Xysticus acerbus (“Xac” peptides). Spiders are natural predators of insects; therefore, their venom has the potential to be insect specific yet still possess a significant degree of structural variety, which may enable venom components to bind or otherwise interact with a spectrum of molecular targets.

The SV toxin peptides of the invention are comprised of at least the following amino acid sequences:

PaspI; SEQ ID NO:1
GCLGEGEKCADWSGPSCCDGFYCSCRSMPYCRCRNNS
PaspII; SEQ ID NO:2
ACVGDGQRCASWSGPYCCDGYYCSCRSMPYCRCRNNS
PaspIII; SEQ ID NO:3
ADCLNEGDWCADWSGPSCCGEMWCSCPGFGKCRCKK
PaspIV; SEQ ID NO:4
ACATKNQRCASWAGPYCCDGFYCSCRSYPGCMCRPNS
XacI; SEQ ID NO:5
ECIGGGGGCSVFSGPSCCGGTCKCKFVLIFPKGCHCT

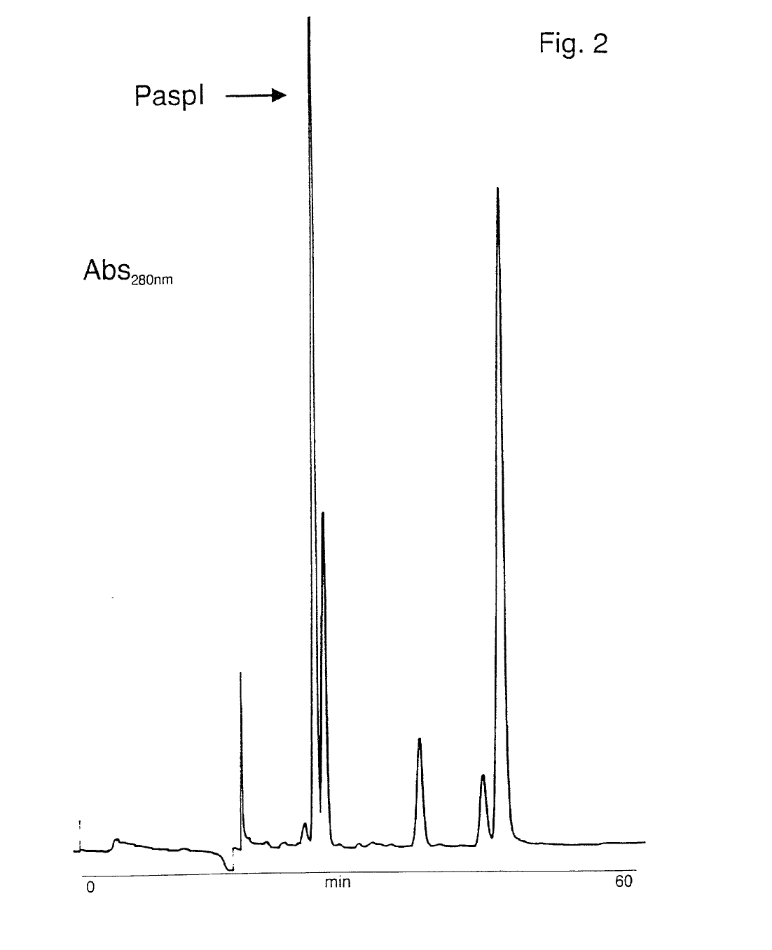

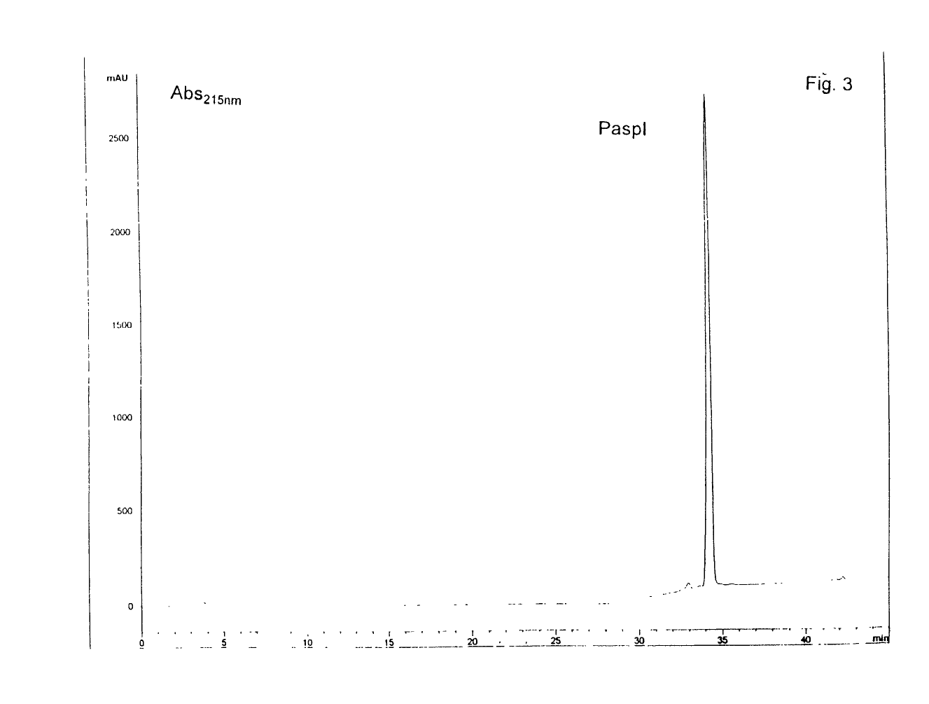

The Pasp and Xac SV toxin peptides described above were isolated from crude spider venom and purified to homogeneity as described in Example 1. The peptides derived from active fractions of the crude venom of Paracoelotes sp. contain 37 (PaspI, PaspII, and PaspII) or 36 (PaspIII) amino acids, with molecular weights ranging from 3.9 to 4.1 Kda (FIGS. 2 and 3).

Pasp SV toxin peptides possess 8 half-cysteines, linked by four disulfide bridges, a structure shared by a number of other spider peptides as part of the generalized conserved motif (X)ₙ,-Cys-(X) ₙ-Cys-(X) ₙ-Cys-Cys-(X) ₙ-Cys-X-Cys—(X) ₙ-Cys-X-Cys-(X) _{n.} This motif is referred to herein as the “cysteine rich toxin motif” and considered to be a hallmark of neurotoxins with diverse ion channel specificities (see, e.g., Omecinsky, et al., Biochemistry, 35:2836-2844, 1996). A core cysteine-rich motif of H₂N-CADWSGPXCCDGXYCSCRSXXXCRCR-COOH (SEQ ID NO:21) is highly conserved between the four SV toxins of SEQ ID NOs:1-4, with some point mutations (X designates variable positions). This variable region found in the SV toxin derived from Paracoelotes sp. is referred to herein as the “Pasp cysteine rich toxin motif”. More variability is observed in both N- and C-terminal regions, which likely confers somewhat different pharmacological properties to each SV toxin peptide.

PaspI is amidated at its C-terminal (PaspI-NH2); however, replacement of the amide with a C-terminal free carboxylic acid (syn-PaspI-OH) does not alter the activity of PaspI, indicating that amidation of the last amino acid is not essential for SV toxin peptide toxicity in this model. The absence of a requirement for an amide at the C-terminus increases the probability for producing an active recombinant PaspI in an insect baculovirus and, more generally, for the commercial scale manufacture of an SV toxin peptide-based biopesticide.

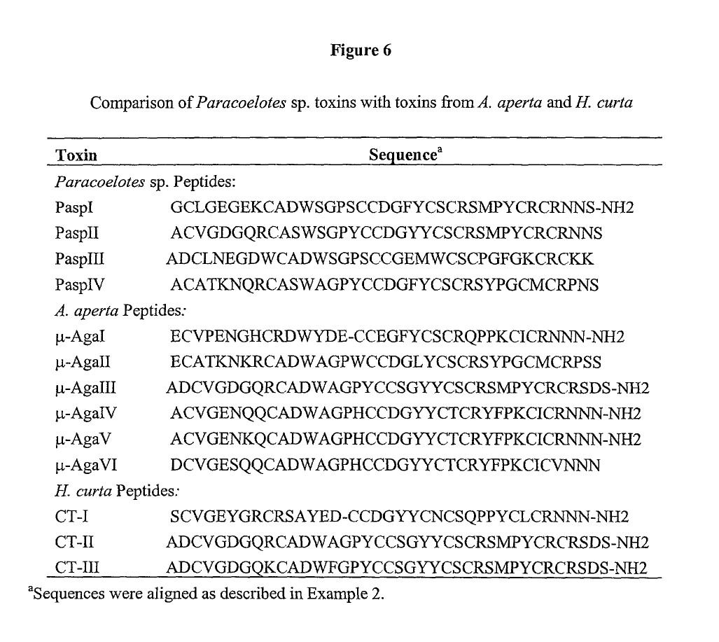

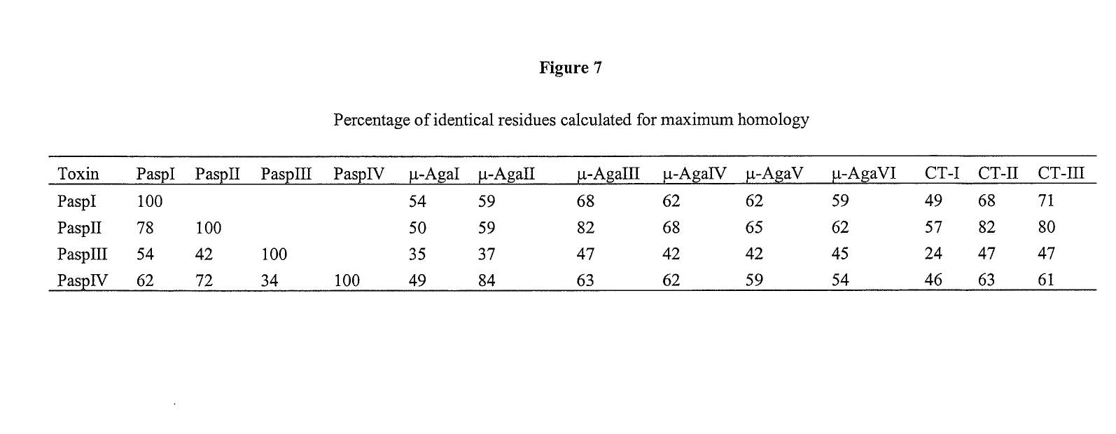

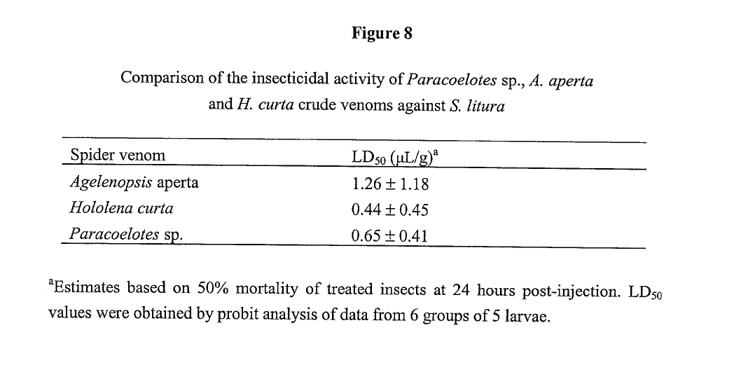

The Pasp SV toxin peptides are also homologous to the previously described insecticidal toxins from Agelenopsis aperta and Hololena curta, each of which exerts some blocking effect on ion channels in the insect nervous system (FIGS. 6 and 7). Given the degree of homology observed in the Pasp SV toxin peptides with the Agelenopsis and Hololena venom peptides, it is likely that the Pasp SV toxin peptides display a similar effect on the insect nervous system. The sequence comparisons and calculations were performed as described in Example 2 and are presented in FIGS. 6 and 7. For reference regarding the activity of venoms from the compared species, those of skill in the art may wish to review, for example, Teramoto et al., Biochem. Biophys. Res. Commun., 196:134-140, 1993 (A. aperta) and Quistad, et al., Toxicon, 29:329-336, 1991 (H. curta). Crude venom extracts from each species were compared for their insecticidal activity in insects as described in Example 3. The Pasp venoms demonstrated comparable, albeit somewhat lower, levels of insecticidal activity than did venom extracts from Agelenopsis aperta and Hololena curta (FIG. 8).

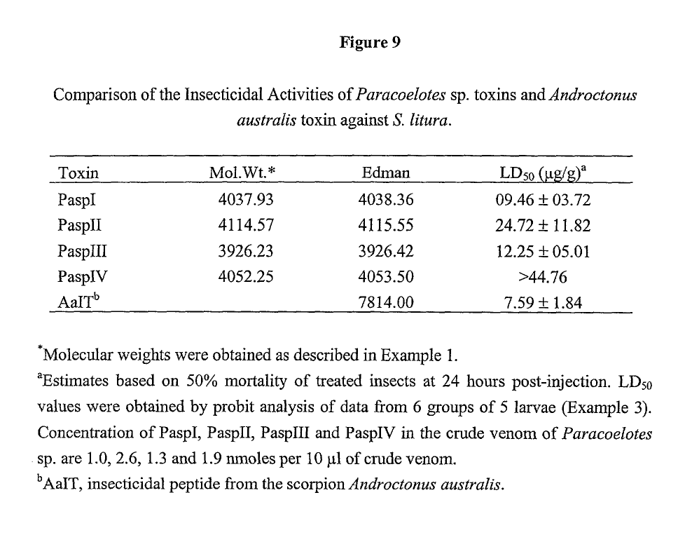

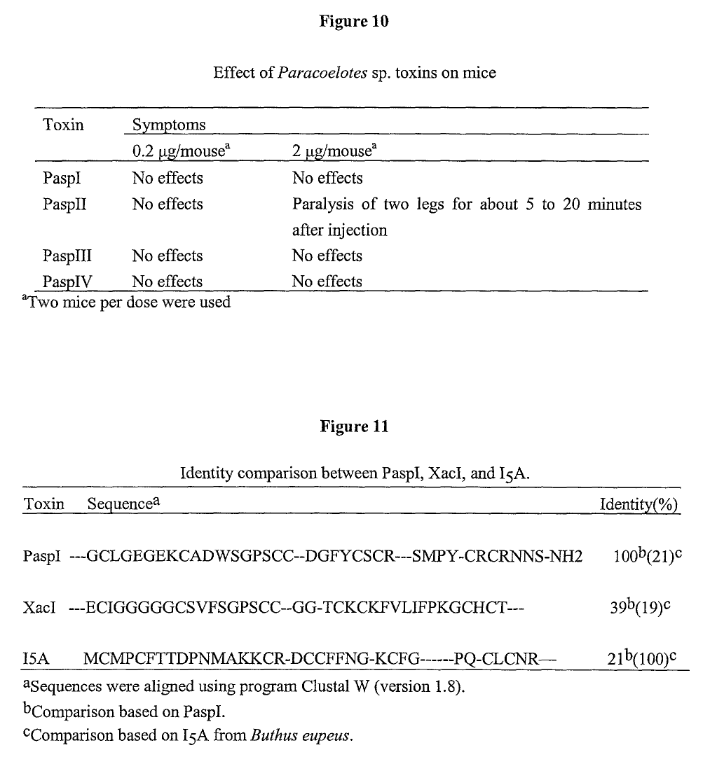

The PaspI, PaspII, PaspIII, PaspIV and XacI SV toxin peptides were also evaluated for insecticidal activity against model insects and mice (FIG. 10). In this respect, PaspI (SEQ ID NO: 1) demonstrated the greatest degree of insecticidal activity against insects, as measured by the dosage at which 50% of the treated test subjects died (LD_{50, 50}% mortality at 24 hours post-injection, following the experimental protocol described in Example 3). Interestingly, the level of insecticidal activity displayed by PaspI was not significantly different from the LD₅₀ measured for toxin AaIT (from the scorpion Androctonus australis; see, FIG. 9), which is one of the most potent insecticidal toxins known to date and has been widely used in genetic engineering of baculoviruses. In contrast to the results obtained in insects, only one of the tested Pasp SV toxin peptides (SEQ ID NOs:1-4) had a toxic effect following intracerebroventricular (ICV) injection in mice at doses of 0.2 and 2 μg per mouse.

Turning to the SV toxin peptide XacI (SEQ ID NO:5), its native source—Xysticus acerbus crude venom extract-is nearly as potent (1.12+0.96 μL/g) in its insecticidal activity as the venom extract from Paracoelotes referred to above. The XacI peptide itself is present in very low concentrations in the crude venom but, once isolated, produces instantaneous paralysis of larvae using the assay procedure described in Example 3. Treated larvae showed some signs of recovery after 8 to 12 hours post-injection, but such recovery may be the effect of a low injection dose, below the LD₅₀. Nonetheless, sequence identity with PaspI (FIG. 11), as well as the observed effect of the peptide on larvae, indicates that XacI exerts insecticidal activity against insects at a level comparable to, if not greater than, the putatively least potent Pasp SV toxin peptide (PaspIV; SEQ ID NO:4).

Isolation And Synthesis Techniques For Use In Obtaining

SV Toxin Peptides, Polynucleotides, Expression Vectors and Antibodies

Natural and Synthetic SV Toxin Peptides

The invention encompasses all of the SV toxin peptides of SEQ ID Nos: 1-5 in any known form that retains the desired insecticidal activity of each peptide; i.e., recombinant, natural and synthetic forms, as well as fragments, analogs and derivatives of such peptides. The terms “fragment,” “derivative” and “analog” when referring to the SV toxin peptides of the invention collectively mean “variant peptides”. Variant SV toxin peptides are those that differ in amino acid sequence but have essentially the same biological function or activity as an SV toxin peptide of SEQ ID NOs:1-5, or more preferably, display insecticidal activity.

SV toxin variant peptides of the present invention are those which have at least 70% similarity (preferably at least 70% identity) to any one of the SV toxin peptides of SEQ ID NOs:1-5, and more preferably at least 80-90% similarity (more preferably at least 80-90% identity) to the SV toxin peptides of SEQ ID NOs:1-5, and still more preferably at least 91%, 92%, 93%, 94%, 95%, 96%, 97%, 98% or 99% similarity (still more preferably at least 91%, 92%, 93%, 94%, 95%, 96%, 97%, 98% or 99% identity) to the SV toxin peptides of SEQ ID NOs:1-5. Preferably, the SV toxin peptide variants comprise a cysteine rich toxin motif. The length of sequence comparison is at least 15 amino acid residues, preferably at least 25 amino acid residues, and more preferably at least 35 amino acid residues.

The present invention also include SV toxin nucleic acids having at least 70% similarity (preferably at least 70% identity) to any one of the SV toxin nucleic acids of SEQ ID NOs:6-10, and more preferably at least 80-90% similarity (more preferably at least 80-90% identity) to the SV toxin nucleic acids of SEQ ID NOs:6-10, and still more preferably at least 91%, 92%, 93%, 94%, 95%, 96%, 97%, 98% or 99% similarity (still more preferably at least 91%, 92%, 93%, 94%, 95%, 96%, 97%, 98% or 99% identity) to the SV toxin peptides of SEQ ID NOs:6-10. As used herein and as known in the art, “similarity” and “identity” between two peptides or nucleic acids is determined by comparing the amino acid or nucleic acid sequence (and with regard to peptides, its conserved amino acid substitutes) of one of the peptides or nucleic acids to the other. Similarity and identity may be determined by procedures that are well known in the art, for example, a BLAST program (Basic Local Alignment Search Tool at the National Center for Biological Information).

More specifically, SV toxin variant peptides include peptides in which:

(i) one or more of the amino acid residues are substituted with a conserved or non-conserved amino acid residue (preferably the former), such as an exchange between the acidic residues Asp and Glu; or,

(ii) one or more of the amino acid residues includes a substituent group; or,

(iii) the mature SV toxin peptide is fused with another compound, such as a compound to increase the half-life of the SV toxin peptide (for example, polyethylene glycol); or,

(iv) additional amino acids are fused to the SV toxin peptide, such as a leader sequence, a secretory sequence, a sequence that is employed for purification of the SV toxin peptide or a proprotein sequence (for subsequent cleavage to release the mature active SV toxin peptide).

Whatever the primary structure of SV toxin peptide variants obtained within the scope of the invention, the insecticidal activity of the SV toxin peptide may be preserved by ensuring that binding domains active in the mature, parent peptide are present in any variants produced. One convenient method for predicting the location of binding domains in a peptide is the coiled-coiled analysis outlined generally in Example 6.

The SV toxin peptides of the present invention are provided in an isolated form, and preferably are purified to homogeneity. The term “isolated” means that the material is removed from its original environment (e.g., the natural environment if it is naturally occurring). For example, a naturally occurring SV toxin peptide present in its natural location in a living animal is not isolated, but the same SV toxin peptide, separated from some or all of the coexisting materials in the natural system, is isolated. Such SV toxin peptides could be part of a composition, and still be isolated in that the composition of which the SV toxin is a part is not itself a part of the toxin's natural environment.

With reference to SEQ ID NOs:1-5, SV toxin peptides and variants thereof can be identified and synthesized by such commonly used methods as t-BOC or FMOC protection of alpha-amino groups. Both methods involve stepwise syntheses whereby a single amino acid is added at each step starting from the C terminus of the peptide (see, Coligan, et al., Current Protocols in Immunology, Wiley Interscience, 1991, Unit 9). Peptides of the invention can also be synthesized by various well known solid phase peptide synthesis methods, such as those described by Merrifield (J. Am. Chem. Soc., 85:2149, 1962), and Stewart and Young (Solid Phase Peptides Synthesis, Freeman, San Francisco, 1969, pp 27-62), using a copolymer (styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with 1% acetic acid solution and then lyophilized to yield the crude material. Purification of the crude material can be acheived by such techniques as gel filtration on a “SEPHADEX G-15” or “SEPHAROSE” affinity column.

Lyophilization of appropriate fractions of the column yield the homogeneous peptide or peptide derivatives, which can then be characterized by such standard techniques as amino acid analysis, thin layer chromatography, high performance liquid chromatography, ultraviolet absorption spectroscopy, molar rotation and solubility, and may also be quantitated by the solid phase Edman degradation.

Recombinant SV Toxin Peptides

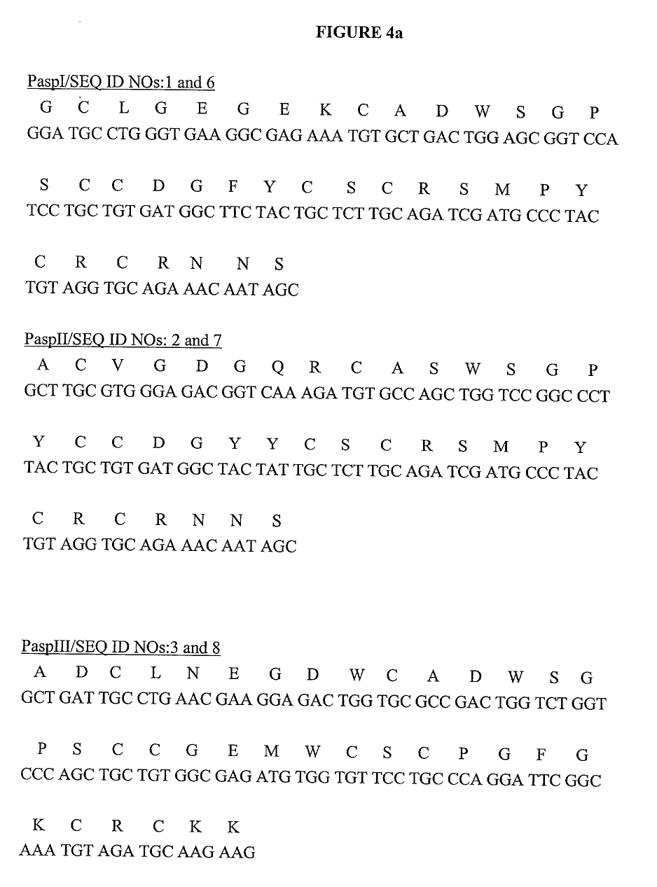

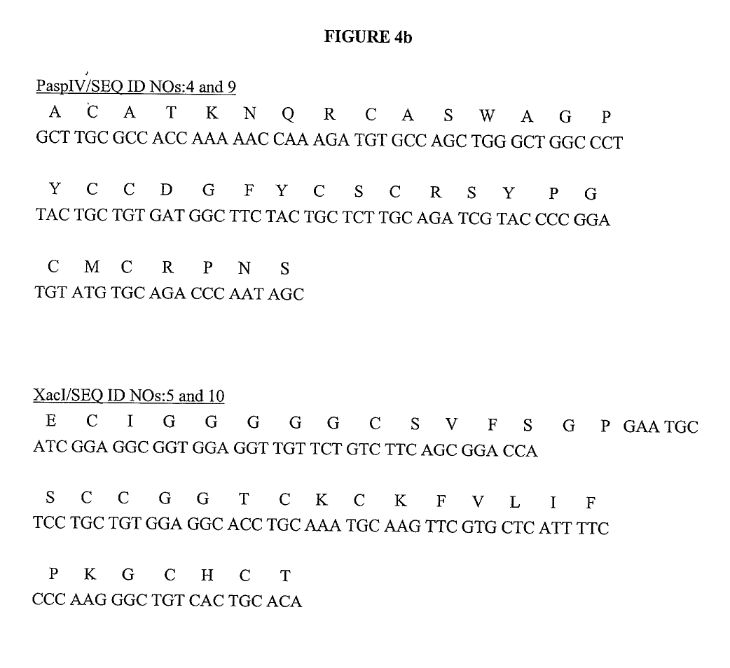

The invention provides polynucleotides that encode the SV toxin peptides of SEQ ID NOs: 1-5, as well as nucleic acids, probes and primers complementary thereto (collectively, “SV toxin polynucleotides” and “SV toxin nucleic acids”). As used herein, “polynucleotide” and “nucleic acid” refer to polymers of deoxyribonucleotides (DNA) or ribonucleotides (RNA), both single-stranded (including sense and antisense strands) and double-stranded, in the form of a separate fragment or as a component of a larger construct. Predicted coding sequences for each of the SV toxin peptides of SEQ ID NOs:1-5 are provided herein as SEQ ID NOs:6-10 and are shown in FIGS. 4a-b.

The nucleotide sequences of SEQ ID NOs.:6-10 are “codon-optimized”. By “codon-optimized”, it is meant that a synthetic polynucleotide is produced which reflects the codon usages of genes derived from the genome of the cell or organism to be used for recombinant protein expression or the codon usages of genes derived from another insect species (such as Drosophila melanogaster or Bombyx mori) which has a sufficient number (at least 10) of known gene sequences. These codon usage tables are used to design the codon optimized gene sequences that code for each SV toxin peptide and for heterologous signal sequences to direct the secretion of the recombinant peptide from host cells. This methodology also permits the preservation, or destruction if desired, of restriction enzyme recognition sites.

Those of ordinary skill will be able to construct suitable codon usage tables for use in designing SV toxin polynucleotide and signal sequence constructs. An especially useful reference in this respect is a database of codon frequency usage for more than 7000 different organisms that is maintained, as of the filing of this application, by the Kazusa DNA Research Institute in Japan and is also available through GENBANK and the World Wide Web at the Internet address dna.affrc.go.jp/˜nakamura/codon.html.

As described further in Example 4, the back translation of SEQ ID NOs: 1-5 which produced the coding sequences of SEQ ID NOs:6-10 was accomplished by applying the standard genetic code and codon usage data from the known protein coding nucleotide sequences of Bombyx mori (and selected to optimize coding for the SV toxin peptide sequences).

Using SEQ ID NOs:6-10 as references, DNA that encodes SV toxin peptides is obtained by chemical synthesis, by screening reverse transcripts of mRNA from insect cells or insect cell lines, or by screening genomic libraries from any cell. For use in the latter contexts, also included within the scope of the invention are nucleic acids that are capable of hybridizing with DNA encoding SV toxin peptides under low stringency conditions (e.g. “primers” or “probes”).

The probes and primers of the invention will generally be oligonucleotides; i.e., either a single stranded polydeoxynucleotide or two complementary polydeoxynucleotide strands that may be chemically synthesized. Such synthetic oligonucleotides have no 5′ phosphate and thus may be labeled with a detectable substance such as a fluorescent group, a radioactive atom or a chemiluminescent group by known methods and used in hybridization assays, such as those described herein.

The invention also encompasses polynucleotides that differ from SEQ ID NOs:6-10. In particular, the invention includes polynucleotides having silent changes (i.e., the change does not or the changes do not alter the amino acid sequence encoded by the polynucleotide) when compared to SEQ ID NOs:6-10. The present invention also relates to nucleotide changes that result in amino acid substitutions, additions, deletions, fusions and truncations of SEQ ID NOs:6-10. In a preferred aspect of the invention, these polypeptides retain the same biological function as the polypeptide shown in SEQ ID NOs:1-5. In particular, the invention encompasses DNA and RNA sequences which are at least 70%, and preferably at least 80%, identical to SV toxin polynucleotides described herein, wherein identity is between, respectively, the bases of the first sequence and the bases of the another sequence, when properly aligned with each other, for example when aligned by BLASTN.

For expression from recombinant expression vectors as detailed further below, SV toxin polynucleotides are modified to include regulatory sequences, such as sequences to direct secretion of a recombinantly produced SV toxin protein from a host cell (e.g., heterologous signal sequences), control region sequences, and selectable marker sequences (e.g., genes coding for dihydrofolate reductase (DHFR), thymidine kinase or neomycin).

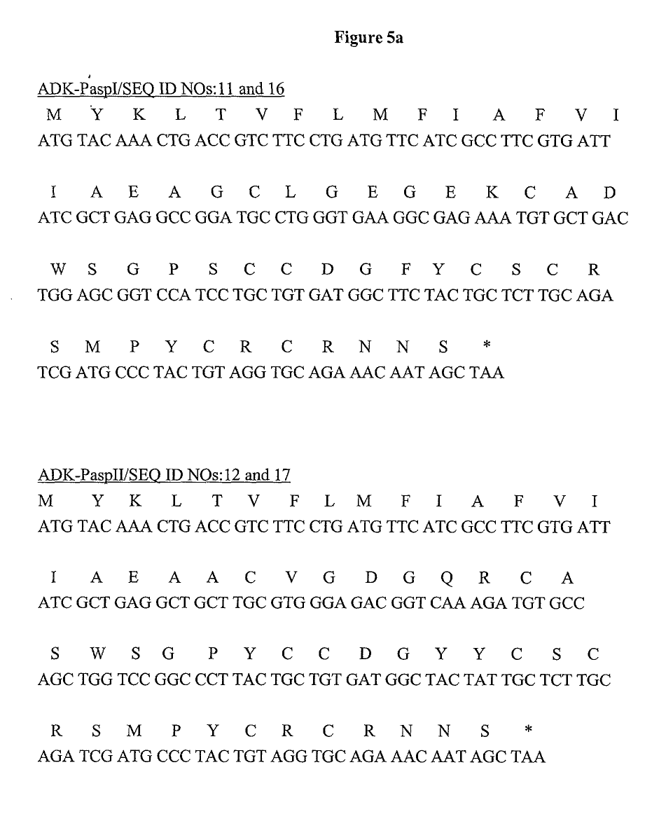

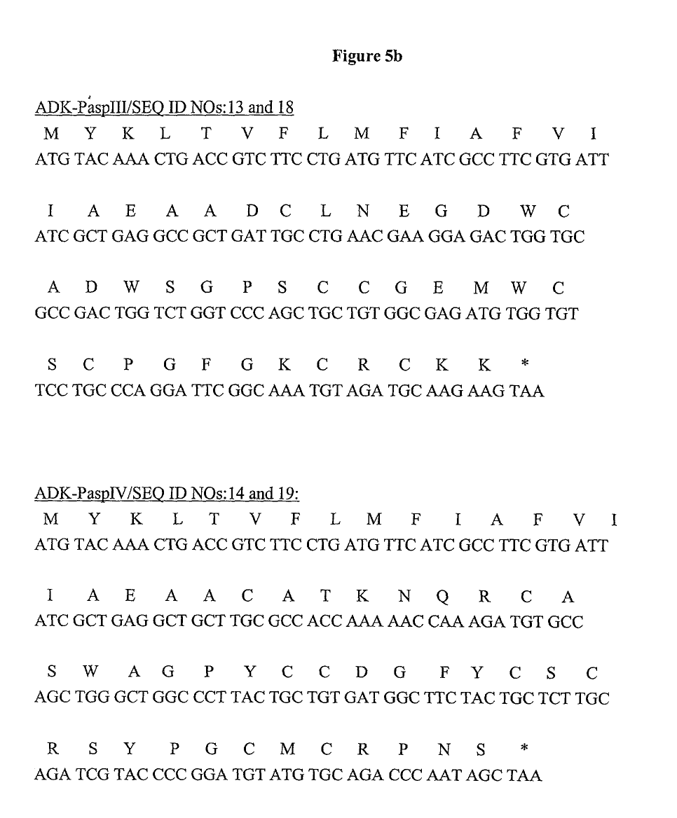

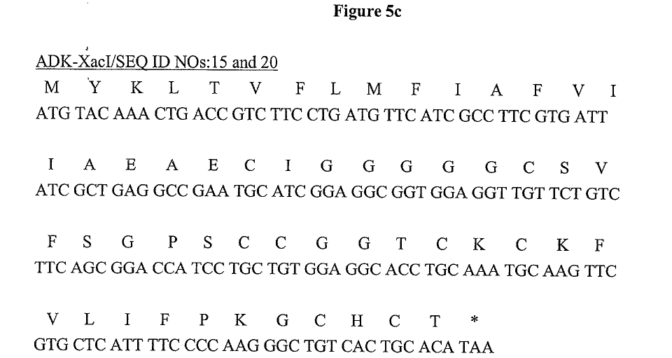

Particularly useful signal sequences for directing the secretion of insect-derived recombinant proteins such as the SV toxin protein described herein are illustrated in U.S. Pat. No. 5,547,871. The U.S. Pat. No. 5,547,871 patent discloses codon-optimized and native sequences for signal sequences. The U.S. Pat. No. 5,547,871 patent discloses the pBMHPC-12 signal sequence from Bombyx mori, the adipokinetic (ADK) hormone signal sequence from Manduca sexta, the apolipophorin signal sequence from Manduca sexta, the chorion signal sequence from Bombyx mori, the cuticle signal sequence from Drosophila melanogaster, the esterase-6 signal sequence from Drosophila melanogaster and the sex specific signal sequence from Bombyx mori. For use in the present invention, each signal sequence is located immediately upstream of either a codon-optimized or a native DNA sequence encoding an SV toxin peptide of the invention. Each such DNA sequence construct is then inserted into an expression vector. FIGS. 5a-c show representative signal peptide-SV amino acid sequences (SEQ ID NOs: 16-15) and their corresponding coding polynucleotide constructs (SEQ ID NOs: 16-20). Each construct utilizes the ADK signal sequence upstream of the SV toxin peptide coding polynucleotide and a TAA termination codon downstream of the coding polynucleotide.

When an expression vector containing such a signal sequence-SV toxin peptide polynucleotide construct is used to transform or infect a suitable host cell, the SV toxin peptide is expressed. The signal sequence assists in the secretion of the SV toxin peptide and is then cleaved off by a signal peptidase, leaving the mature form of the SV toxin peptide.

As used herein, the term “control region” refers to specific sequences at the 5′ and 3′ ends of eukaryotic genes that may be involved in the control of either transcription or translation. Transcription is controlled by specific promoter sequences that contain binding sites for RNA polymerase and auxiliary proteins that modulate RNA polymerase activity. For protein coding genes, the promoter is usually comprised of 50 to several hundred base pairs of DNA located immediately 5′ of the start site of mRNA synthesis. Those of ordinary skill in the art will recognize the importance of promoters in controlling the circumstances, timing and rate of gene transcription. At the 3′ end of most eukaryotic genes is an AATAAA (SEQ ID NO:22) sequence that may be the signal for additional of the poly A tail to the 3′ end of the transcribed mRNA. For use in expression, SV toxin peptide encoding polynucleotides include control region sequences, such as promoters.

Those of ordinary skill in the art are familiar with, or can readily ascertain, the identity of promoters suitable for use with either eukaryotic or prokaryotic expression hosts, such as, for prokaryotic hosts, the β-lactamase and lactose promoter systems (Chang, et al., Nature, 275:615, 1978; and Goeddel, et al., Nature, 281:544, 1979), alkaline phosphatase, the tryptophan (trp) promoter system (Goeddel, Nucleic Acids Res., 8:4057, 1980), the hybrid taq promoter (de Boer, et al., Proc. Natl. Acad. Sci. USA, 80:21-25, 1983); and, for yeast hosts, the promoters for 3-phosphoglycerate kinase (Hitzeman, et al., J Biol. Chem., 255:2073, 1980) or other glycolytic enzymes (Hess, et al. J Adv. Enzyme Reg. 7:149, 1968; and Holland, Biochemistry, 17:4900, 1978) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase; and, for transcription from vectors in mammalian host cells, promoters may be obtained from various sources, for example, the genomes of viruses such as polyoma, Simian Virus 40 (SV40), adenovirus, retroviruses, hepatitis-B virus and cytomegalovirus, or from heterologous mammalian promoters, e.g. beta actin promoter.

For use in expressing SV toxin peptides from transformed insect cells or recombinant baculovirus as referenced below, baculovirus promoters are especially useful. For reference in this regard, those in the art may wish to refer to U.S. Pat. No. 5,162,222, which describes the use of baculovirus early promoters for expression of foreign genes in stably transformed insect cells or recombinant baculovirus. One of ordinary skill in the art is able to ligate promoter sequences to DNA such as the SV toxin polynucleotides of SEQ ID NOs:6-10 using linkers or adapters to supply any required restriction sites.

Recombinant Expression Vectors for use in Delivering and Expressing SV Toxin Peptides of the Invention

The SV toxin polynucleotides of the present invention may be employed for producing SV toxin peptides by recombinant techniques. To this end, an SV toxin polynucleotide may be included in any one of a variety of expression vectors including, without limitation, chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as HIV, vaccinia, adenovirus, fowl pox virus, and pseudorabies. Preferably, insect viruses and cell lines for delivery and expression of the SV toxin peptides of the invention are used.

Baculoviruses are especially well suited for use as expression and delivery vectors for insect control agents. Baculoviruses constitute one of the largest and most diverse groups of insect-pathogenic viruses, and are commonly used as powerful expression systems for heterologous proteins. When used for expression of insecticidal proteins, the time needed to incapacitate a larva is reduced and, when used in conjunction with a signal sequence, the maturation and secretion of a functional SV toxin peptide is facilitated.

An insect virus that is useful for the invention is the baculovirus AcMNPV. Those of skill in the art will recognize that other baculovirus strains may also be used. These include Helicoverpa zea SNPV, Trichoplusia ni MNPV, Rachiplusia ou MNPV, Galleria mellonella MNPV, Spodoptera frugiperda NPV and plaque-purified strains such as the M3, R9, S1 and S3 strains of AcMNPV isolated and characterized in Smith and Summers, J. Virol., 33, 311-319, 1980, as well as Bombyx mori NPV (see also, Smith and Summers, Virol., 89, 517-527, 1978).

Those of ordinary skill in the art will be generally familiar with methods for constructing recombinant baculoviruses in insect cells. For review, reference may be made to Summers and Smith, A Manual of Methods for Baculovirus Vectors and Insect Cell Culture Procedures, Texas Agricultural Experimental Station Bull. No. 1555 (1987). An especially useful method for constructing baculoviruses for use in expression is described in U.S. Application Ser. No. 08/070,164 filed on May 28, 1993, which is published from PCT application US 94/06079, filed May 27, 1994 (referenced in this application as N. Webb et al.). Briefly, according to Webb et al., the signal sequence-SV peptide polynucleotide is cloned into an expression plasmid containing at least a promoter, 5′ UTR and 3′ UTR (including a poly(A)-addition site) and situated so that it is properly oriented for expression between the 5′ and 3′ UTR elements. A DNA fragment containing the assembled expression cassette is excised from the plasmid by digestion with the appropriate restriction endonuclease(s) and inserted into a unique site in the baculovirus genome by DNA fragment ligation in vitro. Depending on the needs of the experiment, the site of insertion may be selected so that none of genes in the baculovirus genome are disrupted by insertion of the expression cassette, or the site may be chosen so that the expression cassette replaces one or a few closely linked genes within the viral genome. Webb et al. discuss both types of design. Following ligation, the recombinant DNA molecules are transfected into insect cells and the recombinant baculoviruses purified by plaque isolation using methods that are familiar to those of ordinary skill in the art. A method for producing a recombinant baculovirus as described by Webb et al. is illustrated in Example 5.

A variety of expression plasmids have been described in the art in addition to those described by Webb et al. For example, a plasmid containing the immediate-early baculovirus promoter IE1 is available through the American Type Culture Collection (ATCC) under Accession Number 40630. Another suitable plasmid which contains the baculovirus delayed-early promoter 35K and the transcriptional enhancer element hr5 is available from the ATCC under Accession Number 40629. The plasmid containing the delayed-early transcripts of the HindIII-k fragments of the baculovirus species Autographa califormica nuclear polyhedron virus (AcMNPV) is also useful in the invention and can be obtained from the ATCC under Accession Number 40628.

The appropriate DNA sequence (e.g., an SV toxin polynucleotide joined to a signal sequence such as those in SEQ ID NOs:16-20) may be inserted into the vector by a variety of procedures. In general, the DNA sequence is inserted into an appropriate restriction endonuclease site(s) by procedures known in the art. Such procedures and others are deemed to be within the scope of those skilled in the art.

Recombinant expression vectors containing an SV toxin polynucleotide and an appropriate promoter or control sequence may be employed to transform an appropriate host to permit the host to express SV toxin peptides. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, Second Edition, Cold Spring Harbor, N.Y., 1989. Representative examples of appropriate hosts for use in recombinant expression are: bacterial cells, such as E. coli, Streptomyces, Bacillus subtilis; fungal cells, such as yeast; insect cells such as Drosophila S2 and Spodoptera Sf9; animal cells such as CHO, COS or Bowes melanoma; adenoviruses; and plant cells. The selection of an appropriate host is deemed to be within the scope of those skilled in the art from the teachings herein.

An especially useful cell line for use in expression of SV toxin peptides is that of Sf9 cells (ATCC accession number CRL171 1), which are derivatives of the cell line designated Spodoptera frugiperda 21 (Sf21). Other insect cell lines that are adequate for propagation of a desirable insect virus include those derived from Trichoplusia ni (TN368), the cabbage looper.

Transcription of SV toxin polynucleotides in eukaryotic cells may be increased by inserting an enhancer sequence into the vector. Enhancers are cis-acting elements of DNA, usually about from 100 to 300 bp that act on a promoter to increase its transcription. Examples include the SV40 enhancer on the late side of the replication origin bp 100 to 270, a cytomegalovirus early promoter enhancer, the polyoma enhancer on the late side of the replication origin, and adenovirus enhancers.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents. Such methods are well known to those skilled in the art.

Recombinantly produced SV toxin peptides can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

In addition to the recombinant expression vectors described herein, the present invention encompasses methods of expressing SV toxin peptides. As described above, a SV toxin nucleic acid is introduced into a host cell and either maintained on a separate plasmid or integrated into the genome of a host cell. In one embodiment of the present invention, a SV toxin nucleic acid described herein is operably linked to a non- SV toxin nucleic acid or a heterologous nucleic acid, both hereinafter referred to as a “chimeric SV toxin nucleic acid sequence.” Within a recombinant expression vector or recombinant expression cassette, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). In one embodiment, the activity of the chimeric SV toxin peptide and those in SEQ ID NOs:1-5 are the same. In other embodiments, the chimeric SV toxin protein encoded by the chimeric altered SV toxin acid sequence has an activity that differs from that of the SV toxin peptide alone.

Antibodies Against SV Toxin Peptides

For use in, for example, the purification of SV toxin peptides, the invention also encompasses polyclonal and monoclonal antibodies that specifically bind to SV toxin peptides. The term “antibody” as used in this invention is meant also to include intact molecules as well as fragments thereof, such as for example, Fab and F(ab′)₂, which are capable of binding the epitopic determinant. Those of ordinary skill in the art will be familiar with techniques for obtaining antibodies to a specific antigen from a biological source, once the antigen is known. As such, extensive details of these techniques will not be set forth here.

Briefly, antibodies with binding specificity for SV toxin peptides can be biologically produced through immunization of a mammal with immunogenic SV toxin peptides (including antigenic fragments thereof and fusion proteins). A multiple injection immunization protocol is preferred for use in immunizing animals with immunogenic SV toxin peptides (see, e.g., Langone, et al., eds., “Production of Antisera with Small Doses of Immunogen: Multiple Intradermal Injections”, Methods of Enzymology, Acad. Press, 1981). For example, a good antibody response can be obtained in rabbits by intradermal injection of 1 mg of immunogenic SV toxin peptides emulsified in Complete Freund's Adjuvant followed several weeks later by one or more boosts of the same antigen in incomplete Freund's Adjuvant.

If desired, immunogenic SV toxin peptides molecules may be coupled to a carrier protein by conjugation using techniques that are well known in the art. Such commonly used carriers that are chemically coupled to the molecules include keyhole limpet hemocyanin (KLH), thyroglobulin, bovine serum albumin (BSA), and tetanus toxoid. The coupled molecule is then used to immunize the animal (e.g., a mouse or a rabbit).

Polyclonal antibodies produced by the immunized animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies (see, for example, Coligan et al., Current Protocols in Immunology, Unit 9, (Wiley Interscience, 1991)).

For preparation of monoclonal antibodies, immunization of a mouse or rat is preferred. The general method used for production of hybridomas secreting monoclonal antibodies (“mAbs”) is well known (Kohler and Milstein, Nature, 256:495, 1975). Briefly, as described by Kohler and Milstein, the technique comprised isolation of lymphocytes from regional draining lymph nodes of five separate cancer patients with either melanoma, teratocarcinoma or cancer of the cervix, glioma or lung. The lymphocytes were obtained from surgical specimens, pooled, and then fused with SHFP-1. Hybridomas were screened for production of antibody that bound to cancer cell lines. An equivalent technique can be used to produce and identify mAbs with specificity for SV toxin peptides.

Confirmation of SV toxin peptide specificity among mAbs can be accomplished using relatively routine screening techniques (such as the enzyme-linked immunosorbent assay, or “ELISA”) to determine the elementary reaction pattern of the mAb of interest.

Bioinsecticide Compositions

Insecticidally useful compositions of SV toxin peptides are prepared/formulated by mixing a peptide of interest with industrially or agriculturally acceptable carriers and inactive components whose identity will be familiar to those of ordinary skill in the art. The formulations of these compositions may be wettable powders, dispersible granular formulations, granules, suspensions, emulsions for aerosols, and other conventional insecticide preparations. An exemplary, not inclusive, list of suitable carriers includes water, alcohol, hydrocarbon or other peptide acceptable organic solvents, or a mineral, animal or vegetable oil, or a powder, such as talc, clay, silicate or kiesaelguhr. Wetting agents, coating agents, UW protectants, dispersants, and sticking agents may also be included. A nutrient, such as a sugar, may be added to increase feeding behavior and/or attract insects. Flow agents such as, for example, clay-based flow agents, may be added to minimize caking of wettable powders or other dry preparations. If desired, the compositions may be formulated as coated particles or as microencapsulated material. The formulations are preferably substantially non-phytotoxic and not detrimental to the integrity of the SV toxin peptides or vector expressing such toxin(s) that is contained therein. Components of these formulations should not deter insect feeding. Exemplary formulations for insecticidal compositions of this invention are disclosed in EP published application 0 697 170 Al; PCT application WO 92/19102; and U.S. Pat. No. 4,498,586. 100731 In alternative embodiments, such compositions may be lyophilized for storage and will be reconstituted according to industrially or agriculturally acceptable means; i.e., suitably prepared and approved for use in the desired application. Advantageously, lyophilized SV toxin peptides can be expected to be fairly stable when stored at room temperature.

The insecticidal compositions of the invention may also include one or more chemical insecticides and/or one or more biological control agents, such as wild type and recombinant insect viruses. Chemical insecticides include without limitation pyrethroids, pyrazolines, organophosphates, carbamates, formadines, and pyrroles, all of which are well known in the art. Exemplary compounds are disclosed in PCT Publication Nos. 96/03048, 96/01055 and 95/95741. Biological control agents include Plutella xylostella baculoviruses (See PCT Publication No. 99/58705 for recombinant forms thereof), Bacillus thurigiensis; Nosema polyvora; M. grnadis; Bracon mellitor; entomopathogenic fungi; and nematodes.

The determination of suitable concentrations and compositions of SV toxin peptide-containing, insecticidal compositions for particular applications will vary with, for example, regulatory requirements, climate, crop identity and insect identity. Thus, such determinations will be made empirically by those of ordinary skill in the art. Methods for Insecticidal Use of SV Toxin Peptides of the Invention

The present invention includes methods of applying insecticidal compositions comprising a carrier and a SV toxin peptide to an insect. The invention also includes methods of controlling an insect population, comprising contacting an insecticide composition with one or more members of the insect population. In preferred embodiments, the insecticide composition comprises a carrier and a spider venom (SV) toxin peptide. In further preferred embodiments, the SV toxin peptide is a Paracoelotes sp. peptide or a Xysticus acerbus peptide and comprises a cysteine rich toxin motif.

As used herein, the term “controlling an insect population” refers to causing the disablement or death of one or more members of the insect population in a given area. In a preferred embodiment, the area is that surrounding a planting of crops or other vegetation. The quantity and frequency of SV toxin peptide application, whether delivered as peptide compositions or via recombinant expression vector application, will necessarily depend on such things as the particular crop being protected, the insect pest and the climate. Accordingly, the quantity and frequency of SV toxin peptide application is best determined empirically, which determination can readily be made by one of ordinary skill in the pest control art.

Methods for Identifying Candidate Insecticide Compounds

Hybridization Screening Assays

SV toxin polynucleotides are useful in screening cultures and libraries for the presence of nucleotide sequences having similarity or identity with SV toxin polynucleotides, which sequences may code for peptides sharing a degree of insecticidal activity with SV toxin peptides. To this end, nucleic acid containing cultures or libraries are screened by hybridization with SV toxin polynucleotides and nucleic acids (e.g., probes).

In general, hybridization is performed under conditions of sufficient stringency to enable one to differentiate between a target SV toxin polynucleotide nucleic acid and other nucleic acids in a sample. The conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2 ×SSC/0.1% SDS at about room temperature (hybridization conditions); 0.2 ×SSC/0.1% SDS at about room temperature (low stringency conditions); 0.2 x SSC/0.1% SDS at about 42° C. (moderate stringency conditions); and 0.1 ×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Screening of Candidate Insecticidal Compounds with SV Toxin Peptides

Binding assays to determine the locus of a SV toxin peptide molecular binding target in insect cells may be performed to determine the identity and location of such target. Such assays may be performed through use of techniques familiar to those of ordinary skill in the art.

Competition and potentiation assays; i.e., wherein a candidate insecticide competes for binding to an SV toxin peptide molecular binding target with an SV toxin peptide, are also useful in elucidating whether such candidate insecticides share or potentiate insecticidal activity with SV toxin peptides. Such assays may be performed using techniques familiar to those of ordinary skill in the art. In particular, peptides and other small molecules may be incubated with target cells and SV toxin peptides, and the relative magnitude of SV toxin peptide activity with and without the presence of the small molecule compared through the steps of:

a. contacting insect cells with a spider venom (SV) toxin peptide selected from the group of SV toxin peptides consisting of the peptides of SEQ ID NOs: 1-5;

b. measuring the response of the insect cells to the SV toxin peptide;

c. contacting insect cells of the same phenotype with a candidate insecticidal compound; and,

d. measuring whether the response of the insect cells following contact with the candidate compound is different from the response measured in step b., wherein such a difference, if detected, indicates that the candidate compound may possess insecticidal activity in that it may compete with the SV toxin peptide for binding to the insect cells, thereby blunting the effect of the SV toxin peptide; or, it may potentiate the activity of the SV toxin peptide, thereby increasing the activity thereof.

Models for use in Confirming Insecticidal Activity of Insecticidal Compounds

Two models for use in evaluating the response of insect cells to candidate insecticidal compounds, including the SV toxin peptides described specifically herein, are described in Example 3. Those of ordinary skill in the art will appreciate that other models for insect responses to candidate insecticides may also be employed to confirm insecticidal activity in a compound. For review in this regard, reference may be made, for example, to Desi, et al., Neurotoxicology, 19:611-616, 1998 (rats); Haas, et al., Chemosphere, 34:699-710, 1997 (mathematical paradigm); Fung et al., Mutat. Res., 374:21-40, 1997 (lacI transgenic mouse mutagenicity assay); and, Guilhermino et al., Ecotoxicol. Environ. Saf., 42:67-74, 1999 (Daphnia magna first-brood chronic bioassay).

All references cited above are incorporated by reference herein for the purpose of illustrating the level of skill in the art with respect to the matter discussed. The practice of the invention is exemplified in the following Examples. Standard abbreviations (e.g., “ml”) are used throughout the Examples unless otherwise noted. The scope of the invention is defined solely by the appended claims, which are in no way limited by the content or scope of the Examples.

### EXAMPLES

### Example 1

Purification and Synthesis of SV Toxin Peptides

A. Preparation of Spider Venoms

Crude venom samples were diluted in distilled water to ten times their initial volume. The crude venoms were first diluted up to ⅔ of their final volume, and then centrifuged (14,000 rpm, 30 min) in the cold room at 4° C. in the a microfuge. After centrifugation the diluted venoms were filtered on 0.45 μm filters. The filters were rinsed with H₂O (⅓ of final volume). Diluted venoms were stored at −20° C.

B. Methods Used to Purify SV Toxin peptides from Spider Venoms

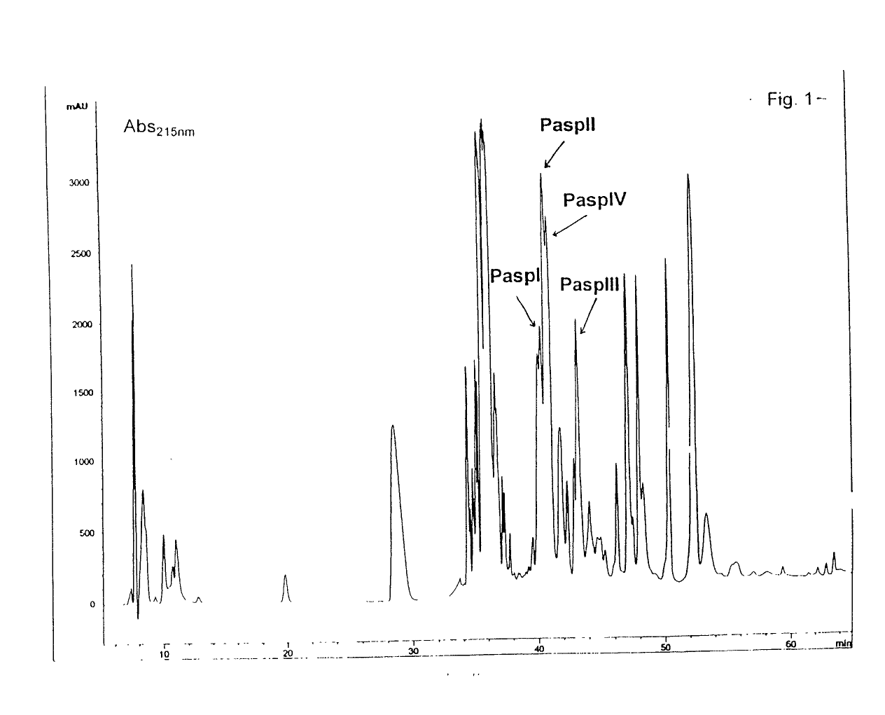

As represented by the purification of PaspI from Paracoelotes s.p., Paracoletes sp. crude venom was fractionated by reversed-phase HPLC on a C₈ column (10×250 mm, Nacalai Tesque, Japan) using a linear gradient of aqueous acetonitrile (CH₃CN) in constant 0.1% trifluoroacetic acid (TFA) at a flow rate of 2 ml/min (FIG. 1). Biologically active peaks were fractionated by cation-exchange chromatography on a TSK-gel SP-5PW column (6×70 mm, Tosoh, Japan) using a linear gradient of from 100 mM acetic acid (pH 2.9) to 1.5 M ammonium acetate in IM acetic acid (pH 4.9) at a flow rate of 1 ml/min (FIG. 2). Finally, PaspI was purified on a C₄ column (4.6×250 mm, Nacalai Tesque) using a linear gradient of aqueous CH₃CN in constant 0.1% TFA at a flow rate of 1 ml/min (FIG. 3).

More specifically, the following protocols were followed to purify all of the SV toxin peptides:

1. Effect of Reversed-Phase HPLC Mobile Phase on Venom Stability

Three microliters of a 10-fold crude venom dilution were diluted in 600 μl of a 60% acetonitrile/0. 1% trifluoroacetic acid (TFA) solution. The mixture was vacuum dried, resuspended in 3 μl of distilled water, and injected into third-instar S. litura larvae.

Controls were done by injection of 3 μl of a 10-fold crude venom dilution diluted in 600 μl distilled water, vacuum dried, resuspended and injected into the larvae. It was determined that the acetonitrile treatment of the venoms had little or no effect on their stability.

2. Reversed-phase HPLC on C8 Columns

The column (Cosmosil semi preparative 5C8-MS, 10×250 mm, Nacalai Tesque) was equilibrated in 0.1% trifluoroacetic acid (TFA) in water. A linear gradient of 0.1% TFA in acetonitrile was applied from 0 to 60% over 60 min at a flow rate of 2 ml/min. Absorbance reading was 215 nm.

3. Cation-Exchange Chromatography

The column (TSK-gel SP-5PW, 6×70 mm, Tosoh) was equilibrated in 100 mM acetic acid (pH 2.9). A linear gradient of 1.5 M ammonium acetate in 1 M acetic acid (pH 4.9) was applied from 0 to 100% over 150 min at a flow rate of 1 ml/min. Absorbance reading was 280 nm.

4. Reversed-Phase HPLC on C4 Columns

The column (Develosil 5C4-MS, 4.6×250 mm, Nomura Chemical) was equilibrated in 0.1% TFA in water. A linear gradient of 0.1% TFA in acetonitrile was applied from 0 to 16% over 16 min, and then from 16 to 30% over 70 min at a flow rate of 1 ml/min. Absorbance reading was 215 nm.

5. Mass Spectrometry of Toxins

For MALDI-TOF analysis, a 10 mg/ml matrix solution of either α-cyano-4-hydroxycinnamic acid (α-CHCA, Aldrich, USA) or 3,5-dimethoxy-4-hydroxycinnamic acid (sinapinic acid—SA, Aldrich, USA) was prepared in 1:1 (α-CHCA) or 2:1 (SA) water/acetonitrile/0.1% TFA. 1 μl of toxin solution was mixed with the matrix (1:10) and 1 μl applied to the sample plate.

Measurements were made on a Perseptive voyager elite system equipped with a 2-meters ion flight tube and delayed-extraction system. Spectra were recorded in linear mode using 337 nm radiation from a nitrogen laser, with a 20 kV acceleration voltage. Mass spectra were obtained by averaging 50-90 scans, using a high laser power setting. Calibration of the system was done prior to each series of measurements using a mixture of β-insulin (3496.94 Da) and bovine insulin (5734.5 Da) and the resulting calibration file used in all subsequent measurements. A new calibration file was generated for each new series. Processing of MS spectra was performed with the GRAMS 386 software on a 486 microcomputer.

C. Synthesis of SV Toxin Peptides

As represented by the synthesis of PaspI, the peptide was synthesized by the solid-phase method by Fmoc methodology on an Applied Biosystems 433-A peptide synthesizer. The 37 amino acid linear peptide was assembled stepwise in a small reactor on 0.48 mmol/mol Fmoc resin. Elongation was performed with N-alpha-Fmoc L-amino acids (1 mmol), by performing cycles of deprotection, washing and coupling. Amino acids were activated by [2-(1H-benzotriazol-1-yl)-1.1.3.3.-tetramethyl-uronium hexafluorophosphate]. Side-chain protecting groups used for trifunctional residues were trityl for Asn, Cys, Gln and His, t-butoxy for Asp and Glu, butyloxycarbonyl for Lys; t-Butyl for Ser, and Ng-2,2,5,7,8 pentamethylchroman-6-sulfonyl for Arg.

After the peptide synthesis was completed, the dried peptidyl resin was treated with 10 trifluoroacetic acid/thioanisol/ethanedithiol/phenol/imidazole/ water (90:4:4:1:0.5:0.5 by weight) per gram of peptidyl resin. After deprotection of the side chain for 3 hours at room temperature, the reaction medium was filtered and precipitated on cold diethyl ether. The precipitate was centrifuged for 1 min at 3000 g, and the collected pellet washed four times with cold diethyl ether. The pellet was dried under a flow of nitrogen to remove residual ether. The pellet was dissolved in an aqueous solution of 20% acetonitrile and the reduced syn-PaspI toxin was separated by reversed phase HPLC on a semi preparative C₈ column (10×250 mm, Nacalai Tesque, Japan).

After fractionation, the eight free cysteine residues were allowed to oxidize by air exposure in an 2 M ammonium aqueous acetate solution (pH 7.2) containing 1 mM reduced glutathione/0.1 mM oxidized glutathione for 24 hours at 5° C. (FIG. 1). The biologically active synthetic toxin was purified by semi preparative reversed-phase and cation-exchange HPLC as previously reported. The identity between the synthetic and natural peptides was verified by capillary electrophoresis, and ESI mass spectrometry.

PaspI, the most potent toxin towards S. litura, was synthesized in two C-terminal forms, carboxylated and amidated. The LD₅₀ of both synthetic toxins towards S. litura were not significantly different (P<0.05) to the native PaspI, indicating that the synthetic toxins contain similar toxicity to the native one. Moreover, after capillary electrophoresis co-elution of PaspI-NH₂. (LD₅₀ 9.7+4.1 μg/g) with the native PaspI-OH (LD₅₀ 9.8±3.9 μg/g) with native PaspI, two peaks were observed. Therefore, it was concluded that the native PaspI was amidated.

### Example 2

Determination of Sequence Homologies of SV Toxin Peptides and Spider Toxin Peptides Having Ion Channel Blocking Capabilities

As shown in FIGS. 4 and 5, the Pasp SV toxin peptides have a significant degree of sequence homology to the well-characterized spider toxins from A. aperta and H. curta, indicating that the Pasp SV toxin peptides may share their ion channel blocking activity. The sequence homology analysis in this respect was performed as follows.

Each SV peptide sequence was used to query all non-redundant GenBank CDS translations and protein sequences in the PDB, Swissprot, PIR and PRF databases for related sequences using version 1.4.11 of the basic local alignment search tool BLASTP (Altschul, Stephen F., Warren Gish, Webb Miller, Eugene W. Myers, and David J. Lipman (1990) J. Mol. Biol. 215:403-10). In each case, the top 6-8 matches were members of the μ-agatoxin group of insect toxins from the funnel web spider Agelenopsis aperta or the closely related curtatoxins from the funnel web spider Hololena curta. The optimum alignment of each SV peptide toxin with each SV peptide toxin, μ-agatoxin and curtatoxin was determined with the FASTP global alignment program, using ktup=1, gap penalties=−12 and −2 and the BLOSUM50 scoring matrix (W. R. Pearson and D. J. Lipman (1988), Proc. Natl. Acad. Sci 85:2444-2448). The percent identity between the two sequences was then calculated as the number of identical residues in the optimum alignment divided by the total number of amino acid positions in the alignment (including internal and terminal gaps) times 100.

### Example 3

Insecticidal Activity of SV Toxin Peptides

Due to the small amounts of material involved, bioassays are realized on a microscale, using a microinjection technique as described below. Drosophila melanogaster was used first as an insect model since it is amenable to injection of volumes of 50 nl volumes or less, and is very sensitive to spider toxins. In order to confirm activity against an economically important crop pest, Spodoptera litura early third-instar larvae were also used. S. litura is a highly polyphagous species, and is considered an international pest on cotton, tobacco, tomato, cabbage, cauliflower, peanuts, beans and other plants. However, S. litura is more resistant to toxins than D. melanogaster, and it requires injection of 10 to 100-fold higher doses. Application of the microinjection technique to S. litura larvae enabled its use as a test insect while lowering the amounts of sample used in the bioassays. In particular, early 3rd instar larvae (3 to 5 mg) can be injected successfully with volumes up to 800 nl of toxin composition. Lethal and paralytic activity of may be evaluated using D. melanogaster adult flies (˜0.7 mg/fly) and S. litura early third-instar larvae (5-8 mg/larva). See Escoubas, P., Pahna, H. F., Nakajima, T. (1995), “A Microinjection technique using Drosophila melanogaster for bioassay-guided isolation of neurotoxins in arthropod venoms”, Toxicon, 33(12), 1549-1555.

To perform the bioassay of SV toxin peptides, SV toxin peptides are injected into the dorsolateral area of Drosophila melanogaster adult flies or into the pronotum of 3^{rd} instar Spodoptera litura larvae using a glass micropipette mounted on an automatic microinjector (A203XVY Nanoliter injector, WPI Precision Instruments, Sarasota, Fla., USA). Flies and larvae are examined five seconds after injection for evidence of involuntary contraction of body segments.

To examine the activity of recombinant baculoviruses expressing SV toxin peptide, each larva is anesthetized with carbon dioxide for 2-5 minutes and then injected with 0.5 μl of diluted virus, using a Hamilton syringe equipped with a 26 gauge needle. The needle is inserted longitudinally between the last two prolegs and then moved anteriorly two to three body segments prior to injection. Following injection, each larva is inspected for the release of dye-stained hemolymph and discarded if sample loss is evident or suspected. The larvae are then stored at 27° C. in covered diet cells (one larva per cell) and inspected visually 2-3 times daily for evidence of morbidity or mortality. An individual is scored as moribund (positive response) if it is unable to right itself within 0.5-2 minutes after being turned on its back.

The oral toxicity of the recombinant SV toxin peptides may be determined by feeding recombinant baculoviruses to second instar Heliothis virescens larvae using a microdrop bioassay procedure as follows. Individual second instar Heliothis virescens larvae are transferred into empty assay wells containing a disk of pre-wetted filter paper. One larva is placed per well. The larvae are then stored for 12-24 hours at 26° C. The next day, a 15 ml aliquot of Stoneville insect diet is heated to boiling. Five mls of water are added to the melted diet. This mixture is reheated to boiling and promptly centrifuged at low speed. Supernatant is removed and a few drops of food dye are added to the clarified supernatant as an aid to visualize the diet. The molten diet is aliquoted into tubes, re-spun in a microfuge, and the clarified supernatant is transferred into new microfuge tubes and cooled to 54° C.

After cooling, the desired amount of PIBs (polyhedral inclusion bodies) may be added and aliquoted onto PARAFILM.™ (American National Can, Greenwich, Conn.), where they harden. The hardened drops are quickly transferred to the wells containing the individual larvae, one drop per larva. The larvae are allowed to feed for 2 hours and those larvae that consume the entire microdrop are placed into a new assay well containing a standard amount of untreated Stoneville diet. Larvae are then placed at 26° C., 50% relative humidity, and monitored twice daily for paralysis and death.

Paralysis is determined by rolling larvae onto their backs and observing for 30 seconds. If the larva remains on its back, it is considered moribund. Moribund larvae generally die during the 24 hours following diagnosis. The duration of the test is generally 8 days. Larvae are inspected two to three times daily and scored for evidence of morbidity and mortality. Both dead and moribund larvae are scored as responding to the treatment.

Intracerebroventricular injections (0.1 111 crude venom-equivalent in 5 μl of 20 mg/ml BSA and 9 mg/ml NaCl) of each toxin were also performed on 20 g male mice (C57B16).

As discussed above and shown in FIGS. 8 and 9, Paracoelotes sp. venom contains four toxic peptides (PaspI, PaspII, PaspIII and PaspIV; SEQ ID NOs:1-4) with insecticidal activity against S. litura third instar larvae. None of the Pasp SV toxin peptides exerted a lethal effect on mice at a dose of 2 micrograms per mouse; however Pasp II did have a toxic effect on mice at the same concentration.

### EXAMPLE 4

Determination of SV Toxin Polynucleotide Coding Sequences

As an initial step in assembling the SV toxin polynucleotide-signal sequence molecules of SEQ ID NOs:16-20, the amino acid sequences of the SV toxin peptides (SEQ ID NOs: 1-5) were reverse translated into DNA sequences representing all possible nucleotide degeneracies (maximum ambiguity) according to the standard genetic code. A list recording the number of times each amino acid occurs in the SV toxin peptide is generated and codons are assigned to each amino acid, with the goal of having the frequency of codon usage in the assembled SV toxin polynucleotide reflect the frequency of codon usage among available protein-coding sequences in the Bombyx mori genome. The codon usage data that served as a guide in assembling the SV toxin polynucleotide sequences is listed in the table below. Particular attention is given to avoiding the use of codons that appear to be used at disproportionately low frequencies in the Bombyx mori genome, such as the codon GGG for glycine and CGG for arginine. Once the initial SV toxin polynucleotide sequence is assembled, it is scanned for the presence of unwanted restriction endonuclease recognition sites, and the sequence is adjusted, as needed, to remove such sites from the final polynucleotide. An example of an unwanted restriction site would be one that might complicate the process of inserting the SV toxin polynucleotide-signal sequence into an expression plasmid or the subsequent insertion of the fully assembled expression cassette into a baculovirus genome, as described by Webb et al. and illustrated in Example 5. Once an acceptable SV toxin polynucleotide is assembled for each SV toxin peptide (e.g., SEQ ID NOs:6-10), it is linked to the codon optimized sequence for the ADK signal peptide to produce the SV toxin polynucleotide-signal sequence molecules of SEQ ID NOs:16-20.

Those of ordinary skill in the art will recognize that the codon usage table is selected to be appropriate for the type of cell or organism that will be used to express the SV toxin peptides and that for any given codon usage table the above procedure can yield a large number of functionally equivalent SV polynucleotide sequences for each SV toxin peptide. The codon usage table utilized in assembling the SV polynucleotide sequences of SEQ ID NOs. 6-10 is set forth below.

C=codon; A=amino acid; x/1000=frequency per 1000 bases; N=number of occurrences

C A x/1000(N)

| | | | |
|---|---|---|---|
| CUU A | 24.0 | (1807) | SEQ ID NO:24 |
| GCC A | 22.8 | (1718) | SEQ ID NO:25 |
| GCA A | 14.0 | (1055) | SEQ ID NO:26 |
| GCG A | 17.0 | (1284) | SEQ ID NO:27 |
| UGU C | 9.9 | (743) | SEQ ID NO:28 |
| UGC C | 14.6 | (1097) | SEQ ID NO:29 |
| GAU D | 23.8 | (1797) | SEQ ID NO:30 |
| GAC D | 30.6 | (2305) | SEQ ID NO:31 |
| GAA E | 32.6 | (2454) | SEQ ID NO:32 |
| GAG E | 28.7 | (2163) | SEQ ID NO:33 |
| UUU F | 12.1 | (913) | SEQ ID NO:34 |
| UUC F | 24.2 | (1826) | SEQ ID NO:35 |
| GGU G | 20.9 | (1574) | SEQ ID NO:36 |
| GGC G | 22.1 | (1668) | SEQ ID NO:37 |
| GGA G | 21.3 | (1607) | SEQ ID NO:38 |
| GGG G | 8.3 | (628) | SEQ ID NO:39 |
| CAU H | 8.1 | (609) | SEQ ID NO:40 |
| CAC H | 13.5 | (1020) | SEQ ID NO:41 |
| AUU I | 17.2 | (1299) | SEQ ID NO:42 |
| AUC I | 22.2 | (1672) | SEQ ID NO:43 |
| AUA I | 13.7 | (1033) | SEQ ID NO:44 |
| AAA K | 30.6 | (2307) | SEQ ID NO:45 |
| AAG K | 27.5 | (2073) | SEQ ID NO:46 |
| UUA L | 11.7 | (883) | SEQ ID NO:47 |
| UUG L | 15.4 | (1161) | SEQ ID NO:48 |
| CUU L | 9.7 | (733) | SEQ ID NO:49 |
| CUC L | 16.8 | (1264) | SEQ ID NO:50 |
| CUA L | 8.5 | (641) | SEQ ID NO:51 |
| CUG L | 18.5 | (1394) | SEQ ID NO:52 |
| AUG M | 24.3 | (1833) | SEQ ID NO:53 |
| AAU N | 18.4 | (1384) | SEQ ID NO:54 |
| AAC N | 25.7 | (1934) | SEQ ID NO:55 |
| CCU P | 12.2 | (919) | SEQ ID NO:56 |
| CCC P | 13.5 | (1020) | SEQ ID NO:57 |
| CCA P | 11.6 | (872) | SEQ ID NO:58 |
| CCG P | 13.3 | (1001) | SEQ ID NO:59 |
| CAA Q | 18.4 | (1384) | SEQ ID NO:60 |
| CAG Q | 18.9 | (1427) | SEQ ID NO:61 |
| CGU R | 8.8 | (661) | SEQ ID NO:62 |
| CGC R | 10.8 | (817) | SEQ ID NO:63 |
| CGA R | 7.2 | (545) | SEQ ID NO:64 |
| CGG R | 6.0 | (451) | SEQ ID NO:65 |
| AGA R | 14.1 | (1061) | SEQ ID NO:66 |
| AGG R | 10.0 | (753) | SEQ ID NO:67 |
| UCU S | 12.8 | (963) | SEQ ID NO:68 |
| UCC S | 13.3 | (1001) | SEQ ID NO:69 |
| UCA S | 10.5 | (790) | SEQ ID NO:70 |
| UCG S | 11.1 | (839) | SEQ ID NO:71 |
| AGU S | 10.1 | (761) | SEQ ID NO:72 |
| AGC S | 14.5 | (1094) | SEQ ID NO:73 |
| ACU T | 14.0 | (1052) | SEQ ID NO:74 |
| ACC T | 15.1 | (1142) | SEQ ID NO:75 |
| ACA T | 14.6 | (1104) | SEQ ID NO:76 |
| ACG T | 11.7 | (879) | SEQ ID NO:77 |
| GUU V | 16.2 | (1224) | SEQ ID NO:78 |
| GUC V | 19.0 | (1436) | SEQ ID NO:79 |
| GUA V | 11.8 | (890) | SEQ ID NO:80 |
| GUG V | 19.5 | (1473) | SEQ ID NO:81 |
| UGG W | 12.0 | (903) | SEQ ID NO:82 |
| UAU Y | 12.4 | (931) | SEQ ID NO:83 |
| UAC Y | 25.3 | (1907) | SEQ ID NO:84 |
| UAA * | 1.3 | (95) | |
| UAG * | 1.0 | (76) | |
| UGA * | 0.4 | (32) | |

### Example 5

Insertion of SV Toxin Polynucleotide-Signal Sequence constructs into Baculovirus Transfer Vectors and Generation of Virus

A. Insertion of SV Toxin Polynucleotide-Signal Sequence into the pMEV1.1 Modular Expression Vector

Webb et al. describes a series of modular expression vectors useful for the insertion of DNA expression cassettes into baculoviruses by DNA ligation in vitro. Each vector is comprised of the following elements cloned into the polylinker region of plasmid pBluescript SK(+) (Stratagene, La Jolla, Calif.): (1) a recognition site for restriction endonuclease Sse8387 I, (2) a DNA module consisting of the promoter and 5′ UTR of an insect cellular gene or a baculovirus gene, (3) a synthetic polylinker containing restriction enzyme recognition sites useful for the insertion of a foreign protein coding sequence, such as the SV toxin polynucleotide-signal sequence, (4) a 3′UTR derived from a baculovirus gene, and (5) a recognition site for restriction endonuclease Bsu36 I. One such plasmid, containing the Autographa californica nucleopolyhedrovirus (AcMNPV) DA26 gene promoter and 5′ UTR and the AcMNPV basic protein gene 3′ UTR is designated pMEV1.1 and is available through the ATCC under Accession Number 69275.

To prepare the SV toxin polynucleotide-signal sequence for insertion into pMEV1.1, a double-stranded synthetic linker sequence containing at least a recognition site for restriction endonuclease BamH I (e.g., 5′-TACGGATCCCATGGTG-3′) (SEQ ID NO:23) is appended onto the 3′ terminus of a SV toxin polynucleotide-signal sequence, resulting in a sequence such as those in SEQ ID NOs:16-20. Those of ordinary skill in the art will be familiar with procedures (e.g., a combination of chemical synthesis of oligonucleotides, DNA ligation and PCR amplification of DNA fragments) that can be used to assemble such a gene fragment de novo. The 5′ end of the fragment is phosphorylated by the action of T4 polynucleotide kinase in the presence of ATP and the product is digested with BamH I, to produce a double-stranded SV toxin polynucleotide-signal sequence fragment that has a blunted terminus at its 5′ end and a 4 nucleotide, BamHI-compatible 5′ overhanging terminus at its 3′ end.

To prepare pMEV1.1 for insertion of the SV toxin polynucleotide-signal sequence, the plasmid is first digested with restriction endonuclease BsmBI, which cuts the DNA at positions −4 (top strand) and 0 (bottom strand) in the 5′ UTR sequence, and then treated with the Klenow fragment of E. coli DNA polymerase I in the presence of the four standard deoxynucleoside triphosphates (dNTPs). This operation produces a blunted terminus at the 3′ end of the 4′ UTR. The DNA is then digested with BamHI, which cuts elsewhere in the polylinker module, and the termini are dephosphorylated by the action of calf intestine alkaline phosphatase. This fragment is ligated with the SV toxin polynucleotide-signal sequence fragment described above to produce a ‘loaded’ pMEV1.1 capable of expressing the SV toxin polynucleotide-signal sequence under the control of the AcMNPV DA26 promoter in insect cells.

B. Insertion of DA26/SV Toxin Polynucleotide-sigNal Sequence Expression Cassette into AcMNPV by DNA Ligation in vitro

Webb et al. also describe how conventional virus construction methods (see Summers and Smith (1987)) are used to insert a synthetic linker fragment containing recognition sites for restriction endonucleases Sse8387 I and Bsu36 I into various non-essential sites in the AcMNPV genome. One such virus, NW6.2.1, contains the Sse8387 I/Bsu36 I linker inserted 92 bp upstream of the start site of translation of the polyhedrin gene. To prepare NW6.2.1 for gene insertion by direct ligation in vitro, the DNA is linearized by sequential digestions with Sse83871 and Bsu36 I, and then separated from the small Bsu-Sse linker fragment by gel filtration chromatography. In a typical experiment forty micrograms of NW6.2.1 viral DNA is digested for 2 hours at 37° C. with 100 units of Sse 8387I (PanVera Corp., Madison, Wis.) in a 250 μl reaction containing 10 mM Tris pH 7.5, 10 mM MgC12, 1 mM dithiothreitol (DTT), 50 mM NaCl, and 0.01% BSA. The reaction mixture is then adjusted to 100 mM NaCl and 50 mM Tris HCl, pH 7.9, and the DNA is digested for 2 hours at 37° C. with 100 units of Bsu 36I (New England Biolabs, Beverly, Mass.). The reaction is terminated by adding SDS to a final concentration of 1%(w/v), NaCl to a final concentration of 0.3 M and EDTA to a concentration of 10 mM. Thereafter, the DNA is chromatographed on a “poly-prep” column (BioRad Laboratories, Richmond, Calif.) containing a 2 ml bed volume of Sephacryl-300 (Pharmacia, Piscataway, N.J.) equilibrated with 10 mM Tris-HCl (pH 8.0), 1 mM EDTA, 0.1% SDS, and 0.3 M NaCl. Twelve 150 μl fractions are collected. Ten microliters of each fraction is analyzed by gel electrophoresis to identify fractions containing the viral DNA. These fractions are pooled, extracted once with phenol:chloroform, and the viral DNA is precipitated with ethanol. The DNA is resuspended in TE (10 mM Tris-HCl (pH 8.0), 1 mM EDTA) at a concentration of 0.2 1 μg/μl and stored at 4° C.

The pMEV1.1/SV polynucleotide-signal sequence plasmid is digested with Bsu36 I and Sse8387 I and the DA26/SV polynucleotide-signal sequence expression cassette is purified by gel electrophoresis. One-half microgram of Bsu36 I/Sse8387 I linearized NW6.2.1 viral DNA is mixed with 12-25 ng of the DA26/SV polynucleotide-signal sequence expression cassette in a 5 μl reaction mixture containing 25 mM Tris-HCl (pH 7.6), 5 mM MgC12, 1 mM ATP, 1 mM DTT, 5% (w/v) polyethylene glycol-8000, and 0.5 units T4 DNA ligase (Gibco-BRL, Gaithersburg, Md.). After an overnight incubation at 16° C., the entire ligation reaction is used to transfect Sf9 cells.

C. Transfection of Insect Cells and Identification of Recombinant Viruses

For transfection, 1.5×10⁶ Sf9 cells are plated in one well of a 6-well cluster dish. After the cells have attached, the cell culture media is replaced with 0.375 ml Grace's insect cell culture medium (Grace, T. D. C. Nature 195:788-789 (1962)). The contents of the ligation reaction are mixed with 0.375 ml of transfection buffer (25 mM HEPES (pH 7.1), 140 mM NaCl, 125 mM CaCl₂) and then added drop wise to the plated cells. The cells are incubated with the DNA for 4 hours at 27° C., washed once with 2 ml of Grace's insect medium supplemented with 0.33% (w/v) lactalbumin hydrolysate, 0.33% (w/v) TC yeastolate, 0.1% (v/v) Pluronic™ F-68 (Gibco BRL, Gaithersburg, Md.) and 10% (v/v) fetal bovine serum (complete TNM-FH medium), and incubated for 72 hours at 27° C. with 2 ml of complete TNM-FH. Viruses in the transfection supernatant are then resolved by standard plaque purification methods (see Smith and Summers (1987)).

To identify the desired recombinants, isolated plaques are picked and used to infect 60,000 Sf9 cells in 0.3 ml complete TNM-FH medium in individual wells of a 48-well cluster plate (Corning Costar, Acton Mass.). After incubating the cells for 3-5 days at 27° C., wells containing the desired recombinant virus are identified by PCR using primers that specifically amplify a defined DNA segment from the SV toxin polynucleotide-signal sequence expression cassette. The PCR procedure is essentially as described by Malitschek and Schartl (Malitschek, B. and M. Schartl. Biotechniques 11:177-178 (1991)). Four microliters of the P1 virus stock is first digested for one hour at 55° C. with 200 μg/ml pronase in a 25 μl reaction containing 1X Buffer A (10 mM Tris (pH 8.3), 50 mM KCl, 0.1 mg/ml gelatin, 0.45% Nonidet™ P40 (Shell Oil Co.), and 0.45% Tween™ 20 (ICI Americas)). The pronase is then inactivated by heating to 95° C. for 12 minutes. For PCR the pronase-treated virus is mixed with 50 pmol of each of the two oligonucleotide primers in a 50 μl reaction containing 200 μM dNTPs, 1.5 mM MgCl₂, 1X Buffer A and 2.5 units AmpliTaq™ DNA polymerase (Perkin-Elmer Cetus, Norwalk, Conn.). The sample is subjected to 25 cycles of amplification, each consisting of 1 min. at 94° C. (denaturation step), 1.5 min. at 55° C. (annealing step), and 2.5 min. at 72° C. (extension step). The 25 cycles are followed by a 7 min. extension step at 72° C. One-fifth of the reaction mix is electrophoresed on a 1.0-1.8% agarose gel to confirm the presence of the desired amplification product. Virus in positive wells is then subjected to one to three additional rounds of plaque purification to ensure genetic purity of the stock.

### Example 6

Coiled-Coil Analysis for Binding Domains in Peptides

Coiled-coil predictions are carried out using the commercially available computer program COILS described by Lupas et al. (Lupas, supra; based on Parry, Biosci. Rep. 2:1017-1024 (1982), the disclosures of which are incorporated herein by reference). The first selection of putative coiled-coil forming domains is done by comparing the protein sequences to an unweighted MTK matrix (Lupas, supra) using a 28 residue scanning window. A second scanning using a 14 residue window is done in order to pinpoint smaller subdomains. The use of a 28 amino acid scanning window permits a more accurate determination of predicted coiled-coil forming regions, but the spatial resolution is poor. On the other hand, a 14 amino acid scanning window provides better resolution, at the expense of a loss in accuracy in the computation of the probabilities.

Predicted coiled-coiled domains are compared with secondary structure predictions obtained using the self-optimized prediction method (SOPM) (Geourjon, et al., Protein Engineering 7:157-164, 1994), which provides a consensus secondary structure from five different prediction methods.

In cases where the calculated consensus sequence shows important discrepancies with the coiled-coil predictions, the Q7-JASEP algorithm is also used (Viswanadhan, et al., Biochemistry 30:11164-11172, 1991). This algorithm combines statistical methods which will be known to those of ordinary skill in the art; to wit, the methods of Chou-Fasman, Nagano, and Burgess-Ponnuswamy-Scheraga, the homology method of Nishikawa, the information theory method of Gamier-Osguthorpe-Robson, and the artificial neural network approach of Qian-Sejnowski.

Using the predictive data obtained from these sources, the locations of putative coiled-coil regions within the SV toxin peptides and variants may be identified.

The invention having been fully described, modifications within its scope will be apparent to those of ordinary skill in the art. All such modifications are within the scope of the invention.

1. An isolated spider venom (SV) toxin peptide, comprising a cysteine rich toxin motif, wherein the peptide is a Paracoelotes sp. peptide or a Xysticus acerbus peptide.

2. The isolated SV toxin peptide of claim 1, wherein the cysteine rich toxin motif is a Pasp cysteine rich toxin motif.

3. The isolated SV toxin peptide of claim 1, wherein the cysteine rich toxin motif comprises an amino acid sequence as shown in SEQ ID NO:21.

4. The isolated SV toxin peptide of claim 1, wherein the SV toxin peptide has insecticidal activity.

5. The isolated SV toxin peptide of claim 1, further comprising a signal peptide.

6. The isolated SV toxin peptide of claim 5, wherein the signal peptide is selected from the group consisting of a cuticle signal peptide from Drosophila melanogaster, a chorion signal peptide from Bombyx mori, a apolipophorin signal peptide from Manduca sexta, a sex specific signal peptide from Bombyx mori, a adipokinetic hormone signal peptide from Manduca sexta, a pBMHPC-12 signal peptide from Bombyx mori and a esterase-6 signal peptide from Drosophila melanogaster.

7. The isolated SV toxin peptide of claim 5, comprising an amino acid sequence selected from the group consisting of SEQ ID NO:11, SEQ ID NO:12, SEQ ID NO:13, SEQ ID NO:14 and SEQ ID NO:15.

8. An isolated spider venom (SV) toxin peptide, comprising an amino acid sequence selected from the group consisting of SEQ ID NO:1, SEQ ID NO:2, SEQ ID NO:3, SEQ ID NO:4, SEQ ID NO:5 and variants having at least 90% identity with SEQ ID NO:1, SEQ ID NO:2, SEQ ID NO:3, SEQ ID NO:4 or SEQ ID NO:5.

9. An insecticide composition, comprising a carrier and the spider venom (SV) toxin peptide of any of claims 1-8.

10. An isolated spider venom (SV) toxin nucleic acid, comprising a nucleotide sequence encoding a cysteine rich toxin motif, wherein the nucleic acid is a Paracoelotes sp. nucleic acid or a Xysticus acerbus nucleic acid.

11. The isolated SV toxin nucleic acid of claim 10, wherein the cysteine rich toxin motif is a Pasp cysteine rich toxin motif.

12. The isolated SV toxin nucleic acid of claim 10, wherein the cysteine rich toxin motif comprises an amino acid sequence as shown in SEQ ID NO:21.

13. The isolated SV toxin nucleic acid of claim 10, wherein the nucleic acid encodes a SV toxin peptide having insecticidal activity.

14. The isolated SV toxin nucleic acid of claim 10, further comprising a signal sequence.

15. The isolated SV toxin nucleic acid of claim 14, wherein the signal sequence is selected from the group consisting of a cuticle signal sequence from Drosophila melanogaster, a chorion signal sequence from Bombyx mori, a apolipophorin signal sequence from Manduca sexta, a sex specific signal sequence from Bombyx mori, a adipokinetic hormone signal sequence from Manduca sexta, a pBMHPC-12 signal sequence from Bombyx mori and a esterase-6 signal sequence from Drosophila melanogaster.

16. The isolated SV toxin nucleic acid of claim 14, comprising a nucleotide sequence selected from the group consisting of SEQ ID NO:16, SEQ ID NO:17, SEQ ID NO:18, SEQ ID NO:19 and SEQ ID NO:20.

17. An isolated spider venom (SV) toxin nucleic acid, comprising a nucleotide sequence encoding a SV toxin peptide selected from the group consisting of SEQ ID NO:1, SEQ ID NO:2, SEQ ID NO:3, SEQ ID NO:4, SEQ ID NO:5 and variants thereof having at least 90% identity with SEQ ID NO:1, SEQ ID NO:2, SEQ ID NO:3, SEQ ID NO:4 or SEQ ID NO:5.

18. An isolated spider venom (SV) toxin nucleic acid, comprising a nucleotide sequence selected from the group consisting of SEQ ID NO:6, SEQ ID NO:7, SEQ ID NO:8, SEQ ID NO:9, SEQ ID NO:10 and variants thereof having at least 90% identity with SEQ ID NO:6, SEQ ID NO:7, SEQ ID NO:8, SEQ ID NO:9, SEQ ID NO:10.

19. An isolated recombinant expression vector, comprising a nucleic acid of any of claims 10-18.

20. The isolated recombinant expression vector of claim 19, wherein the vector is a baculovirus vector.

21. A host cell, comprising the isolated recombinant expression vector of claim 19.

22. A method of expressing an isolated spider venom (SV) toxin peptide, comprising transfecting a recombinant expression vector into a host cell, wherein the vector comprises an isolated spider venom (SV) toxin nucleic acid, and wherein the nucleic acid is a Paracoelotes sp. nucleic acid or a Xysticus acerbus nucleic acid and comprises a nucleotide sequence encoding a cysteine rich toxin motif.

23. The method of claim 22, wherein the cysteine rich toxin motif is a Pasp cysteine rich toxin motif.

24. The method of claim 22, wherein the cysteine rich toxin motif comprises an amino acid sequence as shown in SEQ ID NO:21.

25. The method of claim 22, wherein the SV toxin peptide has insecticidal activity.

26. The method of claim 22, wherein the SV toxin nucleic acid further comprises a signal sequence.

27. The method of claim 26, wherein the signal sequence is selected from the group consisting of a cuticle signal sequence from Drosophila melanogaster, a chorion signal sequence from Bombyx mori, a apolipophorin signal sequence from Manduca sexta, a sex specific signal sequence e from Bombyx mori, a adipokinetic hormone signal sequence e from Manduca sexta, a pBMHPC-12 signal sequence from Bombyx mori and a esterase-6 signal sequence from Drosophila melanogaster.

28. The method of claim 26, wherein the SV toxin peptide comprises an amino acid sequence selected from the group consisting of SEQ ID NO: 1, SEQ ID NO:2, SEQ ID NO:3, SEQ ID NO:4 and SEQ ID NO:5 and variants having at least 90% identity with SEQ ID NO:1, SEQ ID NO:2, SEQ ID NO:3, SEQ ID NO:4 or SEQ ID NO:5.

29. A method of controlling an insect population, comprising contacting an insecticide composition with members of the insect population, wherein the insecticide composition comprises a carrier and a spider venom (SV) toxin peptide, wherein the SV toxin peptide is a Paracoelotes sp. peptide or a Xysticus acerbus peptide and wherein the SV toxin peptide comprises a cysteine rich toxin motif.

30. The method of claim 29, wherein the cysteine rich toxin motif is a Pasp cysteine rich toxin motif.

31. The method of claim 29, wherein the cysteine rich toxin motif comprises an amino acid sequence as shown in SEQ ID NO:21.

32. The method of claim 29, wherein the SV toxin peptide further comprises a signal peptide.

33. The method of claim 32, wherein the signal peptide is selected from the group consisting of a cuticle signal peptide from Drosophila melanogaster, a chorion signal peptide from Bombyx mori, a apolipophorin signal peptide from Manduca sexta, a sex specific signal peptide from Bombyx mori, a adipokinetic hormone signal peptide from Manduca sexta, a pBMHPC-12 signal peptide from Bombyx mori and a esterase-6 signal peptide from Drosophila melanogaster.

34. The method of claim 32, wherein the SV toxin peptide comprises an amino acid sequence selected from the group consisting of SEQ ID NO:11, SEQ ID NO:12, SEQ ID NO:13, SEQ ID NO:14 and SEQ ID NO:15.

35. The method of claim 29, wherein the SV toxin peptide comprises an amino acid sequence selected from the group consisting of SEQ ID NO: 1, SEQ ID NO:2, SEQ ID NO:3, SEQ ID NO:4, SEQ ID NO:5 and variants having at least 90% identity with SEQ ID NO:1, SEQ ID NO:2, SEQ ID NO:3, SEQ ID NO:4 or SEQ ID NO:5.
